(19) United States
(12) Patent Application Publication
(10) Pub. No.: US 2025/0319198 A1
(43) Pub. Date: Oct. 16, 2025

(54) METHOD OF PREPARING STABLE, HIGH-LOAD, WATER-DISPERSIBLE LIPOSOMAL COMPOSITIONS THAT INCORPORATE WATER-INSOLUBLE THERAPEUTICS

(71) Applicant: ESM Technologies, LLC, Carthage, MO (US)

(72) Inventors: Matthew Back, Ozark, MO (US); Kevin J. Ruff, Springfield, MO (US)

(21) Appl. No.: 18/636,428

(22) Filed: Apr. 16, 2024

(51) Int. Cl. A61K 47/69

(52) U.S. Cl. CPC A61K 47/6915

(57) ABSTRACT

The present invention relates to a method of preparing stable, high-load, water-dispersible liposomal compositions that incorporate water-insoluble therapeutics without the use of organic solvents.

### FIELD OF THE INVENTION

The present invention relates to a method of preparing stable, high-load, water-dispersible liposomal compositions that incorporate water-insoluble therapeutics without the use of organic solvents.

### BACKGROUND OF THE INVENTION

Simple liposomes are microscopic spherical structures composed of phospholipids which are amphiphilic in nature, that is, they have long hydrophobic (lipophilic) hydrocarbon chain tails and water-soluble hydrophilic polar heads. Liposomes were first reported in the literature in 1965 [1]. The resulting liposomes therefore encapsulate in their interior a portion of the aqueous medium in which they were formed. This makes liposomes ideal carriers for water-soluble hydrophilic therapeutics such as drugs, mRNA vaccines, or some bioactive natural products (e.g vitamins such as vitamin C or chelated minerals such as magnesium glycinate). However, liposomes can also serve as carriers for hydrophobic therapeutics if sufficient quantities can dissolve in the lipid tail layers that are sandwiched between the two hydrophilic head regions. This has proven more difficult to this point. Simple liposomes with only a single bilayer are referred to as unilamellar, but larger multi-layered liposomes which are essentially liposomes entrapped inside liposomes are referred to as multilamellar. These sizeable liposomes are often formally referred to as large multilamellar vesicles (LMVs). The term “therapeutic” or “therapeutic composition” is intended for the purposes of the present invention to mean any composition or agent administered to a mammal or bird that confers a therapeutic effect to the mammal or bird. The therapeutic composition would be administered topically or orally to the mammal or bird. Uses of the therapeutic composition can include, but are not limited to: as an ingredient in foods, cosmetics, dietary supplements, or pharmaceuticals.

There are numerous methods generally known to those of skill in the art for preparing liposomes, as described by Bhaskarwar, et al. in Liposomes: Fundamentals, Properties, and Applications for Targeted Drug Delivery [2] and by Has and Sunthar in A comprehensive review on recent preparation techniques of liposomes [3]. However, the primary methods for bulk liposome preparation are mechanical aqueous dispersion, thin-film hydration, and solvent-injection dispersion. In mechanical aqueous dispersion, liposome-forming materials are dissolved in a water-soluble organic solvent such as ethanol and this solution is poured into an aqueous medium with mechanical agitation to form liposomes. In thin-film hydration, liposome-forming materials are dissolved in an organic solvent such as ether and the solvent is evaporated while the container is being rotated so that a thin film forms on the surface of the container. This thin film is then hydrated with an aqueous medium, generally in combination with vortexing or sonication, to form liposomes. In solvent-injection dispersion, liposome-forming materials are dissolved in an organic solvent and injected sub-surface via a fine needle into an aqueous medium to form liposomes. The present invention differs from all three of these known primary methods, most notably by excluding the use of any organic solvent(s).

The primary problem created by excluding organic solvents from the liposome-forming process is losing the ability to have all of the components of what will become the liposome in close enough proximity to both form the liposome and entrap the therapeutic component within the liposome in any appreciable concentration. The present invention addresses this critical issue by temporarily solubilizing water-insoluble or poorly soluble therapeutics in the aqueous liposome-forming medium. Key to this temporary solubilization is for the therapeutic compound to possess one or more functional groups with a labile acidic hydrogen or a functional group to which a labile acidic hydrogen can be added, leading to either an anion or a cation, respectively. These functional groups include, but are not limited to: carboxylate (carboxylic acid), enolate (enol), phenolate (phenol), α,β-unsaturated ketone, 1,3-diketone, thiolate (thiol), phosphate, phosphonate, amide, amine, ammonium, and combinations thereof. One of ordinary skill in the art can easily envisage additional functional groups or modifications of the listed functional groups, all of which are intended to be encompassed by the present invention. Natural bioactive compounds that commonly contain these pH labile functional groups include, but are not limited to: alkaloids, carotenoids, flavanols, flavonoids, anthocyanidins, catechins, phytosterols, and polyphenols. Examples of water-insoluble or poorly soluble therapeutics include, but are not limited to: herbal extracts or isolates such as ashwagandha, berberine, boswellia (including boswellic acids), curcumin (including curcuminoids), cannabidiol (CBD) and related cannabinoids, ginseng (including ginsenosides), Ginkgo biloba, ginger, Devil's Claw, sage, St. John's Wort (including hypericin), or turmeric oil; fruit and vegetable extracts or isolates such as astaxanthin, or beta carotene, olive oil unsaponifiables, avocado and soybean unsaponifiables, fisetin, quercetin, resveratrol, or sulforaphane; and vitamins or other metabolic isolates such as biotin, coenzyme Q10 (CoQ10), glutathione, ubiquinol, or ubiquinone. When used throughout, the term “water-insoluble therapeutic” is intended to encompass therapeutics that are also poorly soluble in water. For the purposes of the present invention, the relative term “poorly soluble” will be defined as any compound or mixture of compounds that dissolves in water at a concentration of 5 milligrams per milliliter (mg/ml) or less. One of ordinary skill in the art can easily envisage additional compounds that contain a pH labile functional group rendering them temporarily soluble in an alkaline or acidic aqueous solution, all of which are intended to be encompassed by the present invention.

To provide further background for the problems solved by the present invention, the polyphenolic compound curcumin will be described in significantly greater detail. Curcumin (diferuloyl methane) is the primary polyphenolic constituent in turmeric, a common spice produced from the rhizomes of Curcuma longa L. and related Curcuma plant species that has been used in Asian cooking and as a traditional medicine for centuries. Curcumin, along with its two most prevalent derivatives demethoxycurcumin and bis-demethoxycurcumin, are collectively referred to as curcuminoids. The curcuminoid content of turmeric is only 3-6%, so they have traditionally been extracted, concentrated, and sold as a >95% curcuminoid powder for use in nutritional supplements. Subsequent use of the term “curcumin” will be taken to mean the specific compound curcumin while also including related curcuminoids. Numerous in vitro, in vivo, and human studies have demonstrated the myriad health benefits of curcumin including antioxidant, anti-inflammatory, antibacterial, antifungal, anti-malarial, and anti-cancer properties [4]. However, unformulated curcumin powder is nearly insoluble in water, and is known for its poor gastrointestinal (GI) absorption and subsequent poor bioavailability [5]. Because of its poor absorption, it is difficult for orally administered curcumin compositions to reach blood plasma levels sufficient to exert the aforementioned biological activities. This drastically limits curcumin's usefulness in general healthcare and disease prevention.

Structurally, curcuminoids possess a β-diketone (1,3-diketone) moiety that can exhibit keto-enol tautomerism, with the keto form favored under neutral or acidic pH, which is mostly insoluble in water. When exposed to an alkaline environment the keto-enol equilibrium shifts to favor the enol form, or more precisely the enolate form. The charged enolate anion form is water soluble and the solubility of curcuminoids is directly proportional to the alkalinity of the solution. However, the enolate form of curcumin is highly unstable and degrades relatively quickly to ferulic acid, feruloylmethane, and vanillin. Kumavat, et al. found that at pH 7.4 and 37° C. only 3.83% of a 0.4 μg/mL aqueous solution of curcumin remained after just 60 minutes [6].

Curcuminoids are not only extremely hydrophobic but they also have a low solubility in most edible oils and solvents in relation to typical dosages. This nature of curcuminoids has prevented standard emulsion and liposomal technologies from making compositions containing appreciable amounts of curcuminoids by volume without using strong organic solvents. The present invention is able to overcome these limitations, resulting in a high-load liposomal curcumin composition that is both stable and dispersible in water. Used herein, the term “load” is intended to mean the weight of curcumin (or other water-insoluble therapeutic) per total weight (or volume) of the liposomal composition, that is milligram (mg) of curcumin (or other water-insoluble therapeutic) per gram (g) or milliliter (mL) of liposomal composition. Use of the relative term “high-load” is intended to mean significantly greater than the load found in the prior art. Used herein, the term “stable” is intended to mean BOTH that the liposome particles remain dispersed and do not aggregate or coalesce and that the curcumin (or other water-insoluble therapeutic) within the liposomes does not chemically degrade or break down.

Cheng, et al. prepared curcumin-containing liposomes using a pH-driven, organic solvent-free process [7]. This process differs from the present invention in a number of key points. 1) Cheng, et al. prepared the liposomes prior to adjusting the pH of the mixture, and this process required 4 hours. The method described in the present invention teaches adjusting the pH of the mixture prior to liposome formation, and the process is nearly instantaneous, requiring only a few minutes total (2-5 min.). 2) Cheng, et al. produced curcumin-containing liposomes at a load of 0.4 mg/ml or less. The method described in the present invention produces curcumin-containing liposomes at a load more than 250-fold higher, approximately 100 mg/mL. 3) Cheng, et al. prepared the curcumin-containing liposomes utilizing phospholipids only, with stirring. This likely explains why they were unable to produce high-load liposomes. The method described in the present invention teaches the inclusion of load-enhancing additives, that is, edible oils and proteins, in combination with high-shear mixing.

Barenolz and Haran teach loading of amphiphatic drugs into liposomes through employing a pH gradient [8]. Again, this process differs from the present invention in a number of key points. 1) Barenolz and Haran teach loading the drugs into pre-formed liposomes, wherein the liposomes were formed in the presence of a weak acid (ammonium, NH₄⁺) and the external medium is subsequently altered to be low in ammonium ions thus creating a pH gradient between the inside and outside of the liposomes. The method described in the present invention teaches adjusting the pH of the mixture prior to liposome formation and no pH gradient is utilized during liposome formation. 2) The method taught by Barenolz and Haran utilizes an organic solvent (chloroform), while the method taught in the present invention is organic solvent-free. 3) The method taught by Barenolz and Haran produces liposomes with a load of 5 mg/ml or less of the drug doxorubicin. The method described in the present invention produces curcumin-containing liposomes at a load more than 20-fold higher, approximately 100 mg/mL. 4) Barenolz and Haran teach preparing liposomes with phospholipids and cholesterol and are silent on the inclusion of load-enhancing additives, that is, edible oils and proteins.

Kurzrock, et al. teach a method of producing high-load liposomal curcumin for the treatment of cancer [9]. Again, this process differs from the present invention in a number of key points. 1) The method taught by Kurzrock, et al. utilizes multiple organic solvents (t-butanol and DMSO), while the method taught in the present invention is organic solvent-free. 2) Kurzrock, et al. teach preparing liposomes with phospholipids only and are silent on the inclusion of load-enhancing additives, that is, edible oils and proteins. 3) Kurzrock, et al. are also silent on utilizing a change in pH to influence the solubility of curcumin or other water-insoluble therapeutics.

Hong, et al. teach a method of increasing the drug loading capacity of liposomes through the addition of triglycerides [10]. Some edible oils contain triglycerides, however many also contain diglycerides and monoglycerides. Again, the Hong, et al. process differs from the present invention in a number of key points. 1) The method taught by Hong, et al. utilizes an organic solvent (t-butanol), while the method taught in the present invention is organic solvent-free. 2) Hong, et al. teach preparing liposomes with phospholipids and cholesterol and are silent on the inclusion of other load-enhancing additives, that is, proteins. 3) Hong, et al. are silent on utilizing a change in pH to influence the solubility of water-insoluble therapeutics.

### SUMMARY OF THE INVENTION

In one embodiment of the invention, a method of preparing a liposomal composition containing a water-insoluble or poorly water soluble compound is provided. The method involves the steps of (i) dissolving a water-insoluble or poorly water soluble compound in an alkaline aqueous solution at a final concentration of about 10-100 milligrams per milliliter; (ii) adding an amphiphilic lipid to said dissolved compound; (III) adding a protein to the dissolved compound; (iv) adding an edible oil to the dissolved compound; (v) high-shear mixing the dissolved compound with the addition of amphiphilic lipid, protein and edible oil; and (vi) neutralizing the mixture obtained in step (v).

In another embodiment, the compound possesses one or more functional groups with a labile acidic hydrogen.

According to the invention, one of more functional groups are selected from carboxylate (carboxylic acid), enolate (enol), phenolate (phenol), α,β-unsaturated ketone, 1,3-diketone, thiolate (thiol), phosphate, phosphonate, ammonium, imide, or combinations thereof.

In an embodiment of the invention, the compound is an alkaloid, carotenoid, anthocyanidin, catechin, flavanol, flavonoid, phytosterol, polyphenol, or combination thereof.

In another embodiment, the compound is an herbal extract or isolate, fruit or vegetable extract or isolate, vitamin or metabolic isolate, or combinations thereof.

In another embodiment, the herbal extract or isolate is ashwagandha, boswellia or boswellic acids, curcumin or curcuminoids, cannabidiol (CBD) or related cannabinoids, ginseng or ginsenosides, sage, gingko biloba, ginger, St. John's Wort or hypericin, epigallocatechin gallate (EGCG), or turmeric oil, or combinations thereof.

In yet another embodiment, the fruit or vegetable extract or isolate is astaxanthin, beta carotene, fisetin, quercetin, resveratrol, sulforaphane, avocado or soybean unsaponifiables, or combinations thereof.

In one embodiment, the vitamin or metabolic isolate is biotin, Vitamin D, Vitamin K, coenzyme Q10 (CoQ10), glutathione, retinoic acid, ubiquinol, ubiquinone, or combinations thereof.

In another embodiment, the aqueous solution is made alkaline by adding bases or basic compounds selected from the group consisting of sodium hydroxide, potassium hydroxide, lithium hydroxide, ammonium hydroxide, sodium carbonate, potassium carbonate, calcium carbonate, sodium bicarbonate, potassium bicarbonate, or combinations thereof.

In another embodiment, the amphiphilic lipid is selected from the group consisting of phospholipids, soy lecithin, sunflower lecithin, rapeseed lecithin, egg yolk lecithin, canola lecithin, cottonseed lecithin, phosphatidylcholine and derivatives, phosphatidylinositol and derivatives, phosphatidylethanolamine and phosphatidylserine derivatives, and derivatives, phosphatidylglycerol and derivatives, phosphatidic acid and derivatives, sphingolipids, glycosphingolipids, sphingomyelin and derivatives, or combinations thereof.

In an embodiment, the phosphatidylcholine is distearoylphosphatidylcholine or dimyristoylphosphatidylcholine, and the phosphatidylglycerol is dimyristoylphosphatidylglycerol.

In another embodiment, the protein is selected from the group consisting of whey protein, bone broth, pea protein, sunflower protein, pumpkin seed protein, soybean protein, flaxseed protein, hemp protein, chia seed protein, egg white protein, egg yolk protein, brown rice protein, ovalbumin, casein, bovine serum albumin, collagen, gelatin, or combinations thereof.

In another embodiment, the protein is added at a concentration of about 0.1% to about 5.0% protein.

In an embodiment, the edible oil is selected from the group consisting of sunflower oil, canola oil, safflower oil, soybean oil, corn oil, olive oil, peanut oil, almond oil, flaxseed oil, grapeseed oil, hemp oil, chia seed oil, avocado oil, Ahiflower (Buglossoides arvensis) oil, fish oil, krill oil, medium chain triglyceride (MCT) oil, or combinations thereof.

In yet another embodiment, the edible oil is added at a concentration of about 0.5% to about 20.0% edible oil.

In an embodiment, the method of preparing a liposomal composition containing a water-insoluble or poorly water soluble compound includes an additional step of neutralizing the dissolved compound obtained in step (i) with an acid or an acidic compound selected from hydrochloric acid, sulfuric acid, nitric acid, acetic acid, carbonic acid, citric acid, ascorbic acid, or combinations thereof.

In one embodiment, the dissolved compound obtained in step (i) is acidified to a pH of about 4.6 or less.

In another embodiment, the method of preparing a liposomal composition containing a water-insoluble or poorly water soluble compound includes an additional step of adding a polysaccharide gum to the liposomal composition, where the liposomal composition is selected from locust bean gum, guar gum, gellan gum, xanthan gum, pectin, gum Arabic, agar-agar, carrageenan, alginates, tapioca, methylcellulose, carboxymethylcellulose, cornstarch, potato starch, other starches, or combinations thereof.

In an embodiment, a method of preparing a liposomal composition comprising a water-insoluble or poorly water soluble compound is provided. The method involves the steps of (i) dissolving a water-insoluble or poorly water soluble compound in an acidic aqueous solution at a final concentration of 10-100 milligrams per milliliter; (ii) adding an amphiphilic lipid to said dissolved compound; (III) adding a protein to the dissolved compound; (iv) adding an edible oil to the dissolved compound; (v) high-shear mixing the dissolved compound with the addition of amphiphilic lipid, protein and edible oil; and (vi) neutralizing the mixture obtained in step (v).

In one embodiment, the compound possesses one or more functional groups to which a labile acidic hydrogen can be added.

In another embodiment, the one of more functional groups are selected from amide, imide, amine, pyrrolidine, piperidine, pyridine, indole, imidazole, purine, or combinations thereof.

In yet another embodiment, the compound is selected from alkaloids, carotenoids, anthocyanidins, catechins, flavanols, flavonoids, phytosterols, polyphenols, or combinations thereof.

In an embodiment, the compound is selected from a herbal extract or isolate, a fruit or vegetable extract or isolate, a vitamin or metabolic isolate, or combinations thereof.

In an embodiment, the herbal extract or isolate is ashwagandha, berberine, Boswellia, theobromine, ginseng or ginsenosides, sage, gingko biloba, ginger, St. John's Wort, hypericin, turmeric oil, or combinations thereof.

In another embodiment, the fruit or vegetable extract or isolate is astaxanthin, beta carotene, fisetin, quercetin, resveratrol, sulforaphane, avocado or soybean unsaponifiables, or combinations thereof.

In one embodiment, the vitamin or metabolic isolate is biotin, niacinamide, thiamine, glutathione, or combinations thereof.

In another embodiment, the aqueous solution is made acidic by adding an acid or an acidic compound selected from the group consisting of hydrochloric acid, sulfuric acid, nitric acid, acetic acid, carbonic acid, citric acid, ascorbic acid, or combinations thereof.

In an embodiment, the amphiphilic lipid is selected from phospholipids, soy lecithin, sunflower lecithin, rapeseed lecithin, egg yolk lecithin, canola lecithin, cottonseed lecithin, phosphatidylcholine and derivatives, phosphatidylinositol and derivatives, phosphatidylethanolamine and derivatives, phosphatidylserine and derivatives, phosphatidylglycerol and derivatives, phosphatidic acid and derivatives, sphingolipids, glycosphingolipids, sphingomyelin and derivatives, or combinations thereof.

In one embodiment, the phosphatidylcholine is distearoylphosphatidylcholine or dimyristoylphosphatidylcholine, and the phosphatidylglycerol is dimyristoylphosphatidylglycerol.

In one embodiment, the protein is selected from the group consisting of whey protein, bone broth, pea protein, sunflower protein, pumpkin seed protein, soybean protein, flaxseed protein, hemp protein, chia seed protein, egg white protein, egg yolk protein, brown rice protein, ovalbumin, casein, bovine serum albumin, collagen, gelatin, or combinations thereof.

In another embodiment, the protein is added at a concentration of about 0.1% to about 5.0% protein.

In one embodiment, the edible oil is selected from sunflower oil, canola oil, safflower oil, soybean oil, corn oil, olive oil, peanut oil, almond oil, flaxseed oil, grapeseed oil, hemp oil, chia seed oil, avocado oil, Ahiflower (Buglossoides arvensis) oil, fish oil, krill oil, medium chain triglyceride (MCT) oil, or combinations thereof.

In a further embodiment, the edible oil is added at a concentration of about 0.5% to about 20.0% edible oil.

In another embodiment, the method of preparing a liposomal composition comprising a water-insoluble or poorly water soluble compound includes an additional step of neutralizing the dissolved compound obtained in step (i) with bases or basic compounds selected from the group consisting of sodium hydroxide, potassium hydroxide, lithium hydroxide, ammonium hydroxide, sodium carbonate, potassium carbonate, calcium carbonate, sodium bicarbonate, potassium bicarbonate, or combinations thereof.

In one embodiment, the dissolved compound obtained in step (i) is neutralized to a pH of 4.6 or less.

In one embodiment, the method of preparing a liposomal composition comprising a water-insoluble or poorly water soluble compound includes an additional step of adding a polysaccharide gum to the liposomal composition, wherein the polysaccharide gum is selected from the group consisting of locust bean gum, guar gum, gellan gum, xanthan gum, pectin, gum Arabic, agar-agar, carrageenan, alginates, tapioca, methylcellulose, carboxymethylcellulose, cornstarch, potato starch, other starches, or combinations thereof.

In a final embodiment, the resulting liposomal composition is dried and ground to a fine powder.

### BRIEF DESCRIPTION OF THE FIGURES

FIG. 1: In aqueous media, phospholipids (being amphiphilic) spontaneously organize themselves into self-closing bilayered vesicles due to the interactions of the hydrophilic heads with the aqueous medium and the interactions of the hydrophobic tails with other hydrophobic tails.

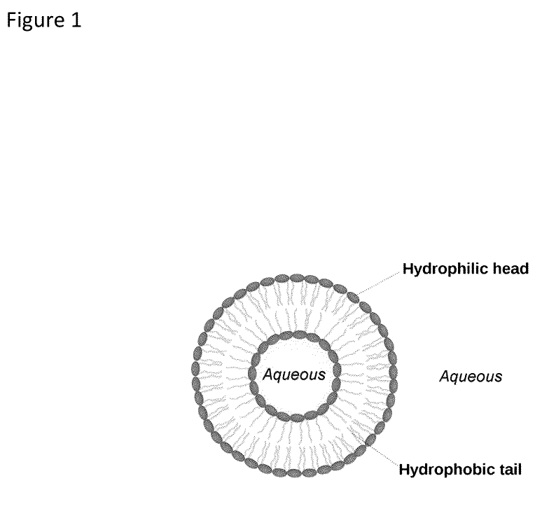

FIG. 2: Structurally, curcuminoids possess a β-diketone (1,3-diketone) moiety that can exhibit keto-enol tautomerism.

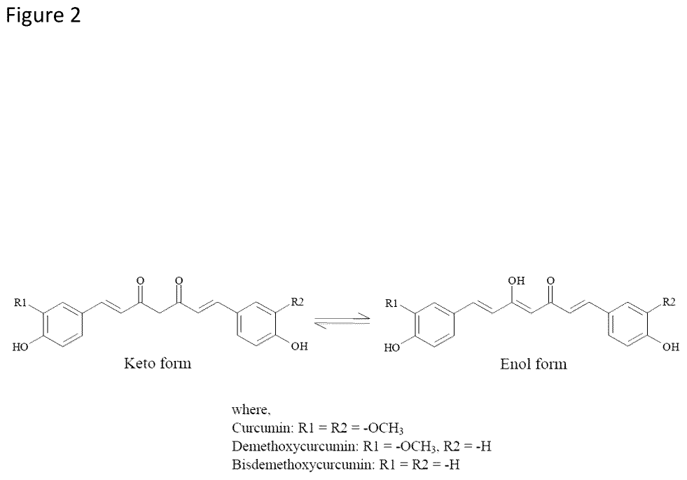

FIG. 3: Grayscale photo of the resulting mixtures showing that all three teachings of the present invention are required to produce stable, high-load liposomal compositions.

FIG. 4: Grayscale photo of the resulting mixtures showing that the addition of protein stabilizes the liposomes, helping them to remain in suspension.

### DETAILED DESCRIPTION OF THE INVENTION

The present invention relates to a method of preparing stable, high-load, water-dispersible liposomal compositions that incorporate water-insoluble therapeutics without the use of organic solvents.

In one aspect of the invention, the aqueous solution is made alkaline with a base or a basic compound. Examples of bases and basic compounds include, but are not limited to: sodium hydroxide, potassium hydroxide, lithium hydroxide, ammonium hydroxide, sodium carbonate, potassium carbonate, calcium carbonate, sodium bicarbonate, and potassium bicarbonate. One of ordinary skill in the art can easily envisage additional compounds that would produce an alkaline solution, that is a pH above 7.0, all of which are intended to be encompassed by the present invention. In another aspect of the invention, the aqueous solution is made acidic with an acid or an acidic compound. Examples of acids and acidic compounds include, but are not limited to: hydrochloric acid, sulfuric acid, nitric acid, acetic acid, carbonic acid, citric acid, and ascorbic acid. One of ordinary skill in the art can easily envisage additional compounds that would produce an acidic solution, that is a pH below 7.0, all of which are intended to be encompassed by the present invention. In a preferred embodiment of the invention, the aqueous solution is made alkaline with food grade sodium hydroxide.

In another aspect of the invention, the water insoluble therapeutic is temporarily solubilized in the alkaline aqueous solution. Examples of water-insoluble or poorly soluble therapeutics include, but are not limited to: herbal extracts or isolates such as ashwagandha, berberine, boswellia (including boswellic acids), curcumin (including curcuminoids), sage, or turmeric oil; fruit and vegetable extracts or isolates such as astaxanthin, or beta carotene, cannabidiol (CBD) or related cannabinoids, fisetin, quercetin, resveratrol, or sulforaphane; and vitamins or other metabolic isolates such as biotin, coenzyme Q10 (CoQ10), glutathione, ubiquinol, or ubiquinone. One of ordinary skill in the art can easily envisage additional compounds that contain a pH labile functional group rendering them temporarily soluble in an alkaline or acidic aqueous solution, all of which are intended to be encompassed by the present invention.

In another aspect of the invention, an amphiphilic lipid compound is employed to form the basis of a liposome. Examples of amphiphilic lipid compounds include, but are not limited to: phospholipids, soy lecithin, sunflower lecithin, rapeseed lecithin, egg yolk lecithin, canola lecithin, cottonseed lecithin, phosphatidylcholine and derivatives (including distearoylphosphatidylcholine and dimyristoylphosphatidylcholine), phosphatidylinositol and derivatives, phosphatidylethanolamine and derivatives, phosphatidylserine and derivatives, phosphatidylglycerol and derivatives (including dimyristoylphosphatidylglycerol), phosphatidic acid and derivatives, sphingolipids, glycosphingolipids, and sphingomyelin and derivatives. One of ordinary skill in the art can easily envisage additional lipid compounds that are amphiphilic, that is both hydrophobic and hydrophilic, all of which are intended to be encompassed by the present invention. In a preferred embodiment of the invention, the amphiphilic lipid compound is food grade sunflower lecithin.

In yet another aspect of the invention, a protein is incorporated into the liposomal mixture. Examples of proteins include, but are not limited to: whey protein, bone broth, pea protein, sunflower protein, pumpkin seed protein, soybean protein, flaxseed protein, hemp protein, chia seed protein, egg white protein, egg yolk protein, brown rice protein, ovalbumin, casein, bovine serum albumin, collagen, and gelatin. One of ordinary skill in the art can easily envisage additional protein powders, all of which are intended to be encompassed by the present invention. In a preferred embodiment of the invention, about 0.1% to about 5.0% protein is incorporated, in a more preferred embodiment about 1.0% to about 2.0%, and in a most preferred embodiment about 1.1%. In a preferred embodiment of the invention, the protein powder is food grade whey protein.

In a further aspect of the invention, an edible oil is incorporated into the liposomal mixture. Examples of edible oils include, but are not limited to: sunflower oil, canola oil, safflower oil, soybean oil, corn oil, olive oil, peanut oil, almond oil, flaxseed oil, grapeseed oil, hemp oil, chia seed oil, avocado oil, Ahiflower (Buglossoides arvensis) oil, fish oil, krill oil, and medium chain triglyceride (MCT) oil. One of ordinary skill in the art can easily envisage additional edible oils, all of which are intended to be encompassed by the present invention. In a preferred embodiment of the invention, about 0.5% to about 20.0% edible oil is incorporated, in a more preferred embodiment about 2.0% to about 6.0%, and in a most preferred embodiment about 2.3%. In a preferred embodiment of the invention, the edible oil is food grade MCT oil.

In another aspect of the invention, the alkaline liposomal mixture is neutralized with an acid or the acidic liposomal mixture is neutralized with a base. Examples of acids and acidic compounds that can be used to neutralize the liposomal mixture include, but are not limited to: hydrochloric acid, sulfuric acid, nitric acid, acetic acid, carbonic acid, citric acid, and ascorbic acid. One of ordinary skill in the art can easily envisage additional compounds that would neutralize an alkaline mixture, that is produce a final pH of about 7.0, all of which are intended to be encompassed by the present invention. Examples of bases and basic compounds that can be used to neutralize the liposomal mixture include, but are not limited to: sodium hydroxide, potassium hydroxide, lithium hydroxide, ammonium hydroxide, sodium carbonate, potassium carbonate, calcium carbonate, sodium bicarbonate, and potassium bicarbonate. One of ordinary skill in the art can easily envisage additional compounds that would neutralize an acidic mixture, that is produce a final pH of about 7.0, all of which are intended to be encompassed by the present invention. In a preferred embodiment of the invention, the acid used to neutralize the alkaline liposomal mixture is food grade citric acid. In a more preferred embodiment of the invention, the alkaline liposomal mixture is adjusted beyond neutral, below about pH 4.6, which meets the U.S. Food & Drug Administration definition of an acidified food.

In another aspect of the invention, a polysaccharide gum or a combination of polysaccharide gums can also be incorporated to help stabilize the liposomal suspension for longer periods of time. Examples of polysaccharide gums include, but are not limited to: locust bean gum, guar gum, gellan gum, xanthan gum, pectin, gum Arabic, agar-agar, carrageenan, alginates, tapioca, methylcellulose, carboxymethylcellulose, cornstarch, potato starch, and other starches. One of ordinary skill in the art can easily envisage additional compounds that would thicken or stabilize the liposomal mixture, all of which are intended to be encompassed by the present invention. In a preferred embodiment of the invention, a mixture of guar gum and xanthan gum is employed to stabilize the liposomal suspension for up to 1 year at room temperature.

In a further aspect of the invention, the resulting liposomal mixture can be dried and ground to a powder. Numerous methods of drying are known in the art and all of these methods are intended to be encompassed by the present invention. A simple method of drying would be to place the liposomal mixture in a suitable (heat stable) container in an oven for a time period sufficient to remove nearly all of the water present. Another method of drying contemplated in the present invention would be to spray-dry the liposomal mixture. Numerous methods of grinding are also known in the art and all of these methods are intended to be encompassed by the present invention.

### EXAMPLES

The following non-limiting examples have been carried out to illustrate various embodiments of the invention:

### Example 1: Preparation of a High-Load Liposomal Curcumin Composition

The following example is illustrative of the preparation of a high-load liposomal curcumin composition at a load of 6.7% curcuminoids (67.3 mg/g of mixture). Add 77.79 g of potable water to a suitable vessel. Add 2.69 g of food grade sodium hydroxide (NaOH) pellets and dissolve. While mixing at approximately 1,000-2,000 RPM with a high-shear mixer, add 6.73 g of 95% curcuminoids powder and mix until fully dissolved. The curcuminoids will turn a deep amber red during this time, due to the alkaline pH of the solution. While mixing at approximately 4,000-5,000 RPM, add 2.96 g of food grade sunflower lecithin and 1.1 g of food grade whey protein. Once the mixture is fully homogenized, the high-shear mixer speed is increased to approximately 12,000-15,000 RPM and 2.28 g of food grade MCT oil is slowly added over ˜1 minute. Immediately begin slowly adding 6.06 g of food grade citric acid over 2-3 minutes, slowing or stopping the addition of the acid as the color begins to change from amber to yellow-orange, because the mixture becomes very viscous temporarily. As the mixture becomes less viscous resume the citric acid addition. Following acid addition, continue to mix at approximately 12,000-15,000 RPM until the final bright yellow color occurs. Finally, add 100 mg each of food grade xanthan and guar gums.

### Example 2: Preparation of High-Load Liposomal Curcumin Compositions without A) Acid/Base Adjustment, B) Protein Additive, or C) Edible Oil Additive

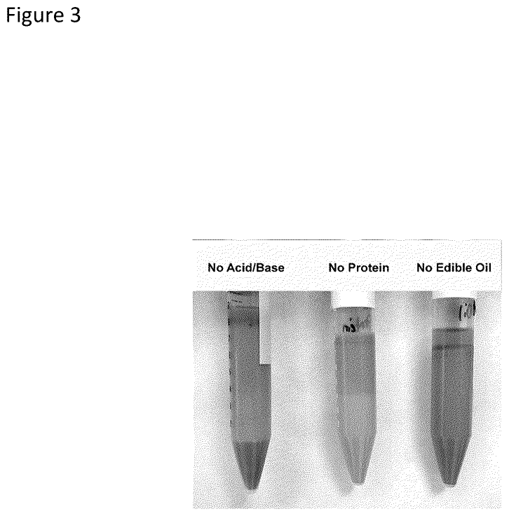

The following example is illustrative of the preparation of a high-load liposomal curcumin compositions without the teachings of the present invention. Liposomal curcumin compositions were prepared as in Example 1 without the inclusion of either A) acid/base adjustment, B) protein additive, or C) edible oil additive. The resulting liposomal compositions were centrifuged at 5,000×g for about 10 minutes. FIG. 3 is a grayscale photo of the resulting mixtures showing that all three teachings of the present invention are required to produce stable, high-load liposomal compositions. Without acid/base adjustment, the water-insoluble curcumin is poorly entrapped in the liposomes and curcumin powder collects at the bottom of the centrifuge tube (dark area in the conical portion). Without protein additive the liposomes are unstable and poorly entrap the water-insoluble curcumin, resulting in two separate phases within the centrifuge tube (lighter area at the bottom and darker area at the top). Without edible oil additive the liposomes are unstable and poorly entrap the water-insoluble curcumin, resulting in two separate phases within the centrifuge tube (clear aqueous layer at the top and darker area at the bottom).

### Example 3: Determination of Preferred Edible Oils for the Preparation of Liposomal Compositions

Liposomal curcumin compositions were prepared as in Example 1 while varying the choice of edible oil. The more preferred edible oils, for example those with a greater number of “+” after them, produce fully homogenous mixtures that are smooth and creamy. The poorly performing edible oils, that is those with fewer “+” after them, produce inhomogeneous mixtures that may separate into layers of oil and water, or may result in precipitation of unencapsulated curcumin, or may be grainy or granular (from incomplete or poor encapsulation of curcumin), or the viscosity of the mixture becomes such that mixing is difficult, or any combination of these.

| Edible Oils Evaluated | Rating |
|---|---|
| Medium Chain | +++++ |
| Triglycerides (MCT) oil | |
| Fish oil | ++++ |
| Canola oil | ++ |
| Soybean oil | ++ |
| Avocado oil | ++ |
| Olive oil | ++ |
| Grapeseed oil | ++ |
| Sunflower oil | + |
| Safflower oil | + |
| Corn oil | + |
| AhiFlower oil | + |
| Hemp oil | + |
| Peanut oil | + |
| Almond oil | + |
| Flaxseed oil | + |

### Example 4: Determination of Preferred Proteins for the Preparation of Liposomal Compositions

Liposomal curcumin compositions were prepared as in Example 1 while varying the choice of protein. The more preferred proteins, that is those with a greater number of “+” after them, produce fully homogenous mixtures that are smooth and creamy. The poorly performing proteins, that is those with fewer “+” after them, produce inhomogeneous mixtures that may be grainy or granular (from incomplete or poor encapsulation of curcumin), or the viscosity of the mixture becomes such that mixing is difficult, or any combination of these.

| Proteins Evaluated | Rating |
|---|---|
| Whey protein | +++++ |
| Beef Bone Broth | +++++ |
| Ovalbumin | +++++ |
| Pea protein | ++++ |
| Sunflower protein | ++++ |
| Pumpkin seed protein | ++++ |
| Soy protein | +++ |
| Flax protein | +++ |
| Hemp Protein | +++ |
| Casein | +++ |
| Collagen | ++ |
| Egg white protein | ++ |
| Egg yolk protein | + |
| Gelatin | + |
| Brown Rice protein | + |
| Chia seed protein | + |

### Example 5: Determination of a Working Range for the Inclusion of Edible Oils

Liposomal curcumin compositions were prepared as in Example 1 while varying the amount by weight percent of the edible oil, medium chain triglycerides (MCT) oil. The amount by weight of water included in the preparation was reduced to maintain a constant overall concentration for all other components of the liposomal mixture. This resulted in a working 10 range of about 0.5% to about 20.0% inclusion of edible oil, with a preferred inclusion of about 2.0% to about 6.0%, and a more preferred inclusion of about 2.3%.

| Weight % of | |
|---|---|
| MCT Oil | Rating |
| 0% | − |
| 0.5% | + |
| 1.0% | ++ |
| 1.5% | +++ |
| 2.0% | +++++ |
| 2.5% | +++++ |
| 3.0% | +++++ |
| 3.5% | +++++ |
| 4.0% | +++++ |
| 6.0% | +++++ |
| 10.0% | ++++ |
| 15.0% | +++ |
| 20.0% | ++ |

### Example 6: Determination of a Working Range for the Inclusion of Proteins

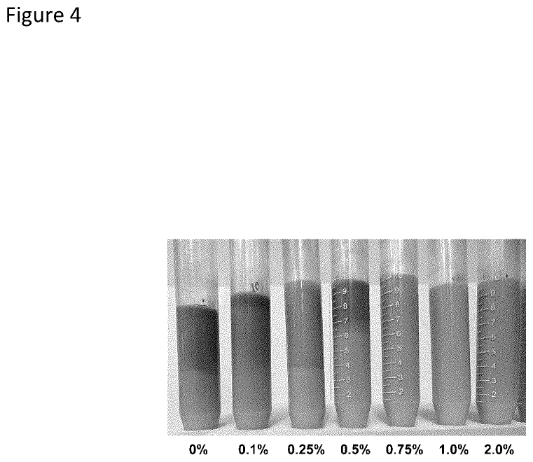

Liposomal curcumin compositions were prepared as in Example 1 while varying the amount by weight percent of the protein, pea protein. The amount by weight of water included in the preparation was reduced to maintain a constant overall concentration for all other components of the liposomal mixture. The liposomal compositions were centrifuged at 5,000×g for 10 minutes. FIG. 4 is a grayscale photo of the resulting mixtures showing that the addition of protein stabilizes the liposomes, helping them to remain in suspension. This results in a working range of about 0.1% to about 5.0% inclusion of protein, with a preferred inclusion of about 1.0% to about 2.0%, and a more preferred inclusion of about 1.1%.

| Weight % of | |
|---|---|
| Pea Protein | Rating |
| 0% | − |
| 0.10% | + |
| 0.25% | + |
| 0.50% | ++ |
| 0.75% | +++ |
| 1.00% | ++++ |
| 1.25% | +++++ |
| 1.50% | +++++ |
| 2.00% | +++++ |
| 5.00% | +++ |
| 10.0% | − |

### Example 7: Preparation of a Variety of High-Load Liposomal Compositions with Different Water-Insoluble Therapeutics

The following examples are illustrative of the broad applicability of the present invention and incorporate water-insoluble therapeutics with a variety of acid-labile functional groups. Curcumin contains a 1,3-diketone moiety, quercetin and resveratrol both contain multiple acid-labile phenolic groups, Ashwagandha compounds and coenzyme Q10 contain α,β-unsaturated ketones, and sage extract and Boswellia extract contain carboxylic acids. Liposomal compositions were prepared as in Example 1 while varying the water-insoluble therapeutic (in place of the curcumin). The ratios of the other components of the liposomal composition were maintained.

| Compounds | |
|---|---|
| Evaluated | Rating |
| Curcumin | +++++ |
| Quercetin | +++++ |
| Resveratrol | ++++ |
| Boswellia extract | ++++ |
| Ashwagandha | ++++ |
| Sage extract | ++++ |
| CoQ10 | +++ |
| Beta Carotene | +++ |
| Astaxanthin | + |
| Lutein | + |

### Example 8: Drying to a Powder and Reconstituting a High-Load Liposomal Curcumin Composition

The following example is illustrative of the capacity of the high-load liposomal composition to be dried to a powder and then reconstituted in water without destruction of its liposomal characteristics (e.g. forming and maintaining a suspension). A liposomal curcumin composition was prepared as in Example 1 without adding the xanthan and guar gums. The composition was then centrifuged at 5,000×g for about 30-40 minutes. The supernatant liquid was decanted taking care to reserve the solids in the bottom of the vessel. The wet solids were removed from the vessel and dried in a thin layer in an oven at 60-70° C. for 2-3 hours with a gentle air stream sweep. The coarse material was then ground in a mechanical grinder to produce a fine powder. The fine powder was reconstituted in ˜75 mL of purified water. The resulting reconstituted liposomal composition had the same physical properties as prior to drying and grinding. That is, it produced a stable suspension without curcumin precipitating out.

### REFERENCES

[1] A. D. Bangham, M. M. Standish and J. C. Watkins, “Diffusion of univalent ions across the lamellae of swollen phospholipids,” J Mol Biol, vol. 13, no. 1, pp. 238-252, 1965.
[2] M. Bhaskarwar, A. Joshi and A. N. Bhaskarwar, Liposomes: Fundamentals, Properties and Applications for Targeted Drug Delivery, New York: Momentum Press, 2019.
[3] C. Has and P. Sunthar, “A comprehensive review on recent preparation techniques of liposomes,” J Liposome Res, vol. 30, no. 4, p. 336-365, 2020.
[4] S. J. Hewlings and D. S. Kalman, “Curcumin: A Review of Its' Effects on Human Health,” Foods, vol. 6, p. 92, 2017.
[5] V. Ravindranath and N. Chandrasekhara, “Absorption and Tissue Distribution of Curcumin in Rats,” Toxicol, vol. 16, pp. 259-265, 1980.
[6] S. D. Kumavat, Y. S. Chaudhari, P. Borole, P. Mishra, K. Shenghani and P. Duvvuri, “Degradation Studies of Curcumin,” Int J Pharmacy Rev Res, vol. 3, no. 2, pp. 50-55, 2013.
[7] C. Cheng, S. Peng, Z. Li, L. Zou, W. Liu and C. Liua, “Improved bioavailability of curcumin in liposomes prepared using a pH-driven, organic solvent-free, easily scalable process,” RSC Adv, vol. 7, pp. 25978-25986, 2017.
[8] Y. Barenolz and G. Haran, “Method of Amphiphatic Drug loading in Liposomes by pH Gradient”. U.S. Pat. No. 5,192,549, 9 Mar. 1993.
[9] K. R, L. Li, K. Mehta and B. B. Aggarwal, “Liposomal Curcumin for Treatment of Cancer”. U.S. Pat. No. 9,283,185, 15 Mar. 2016.
[10] S. Hong, S. H. Kim and S. Lim, “Effects of triglycerides on the hydrophobic drug loading capacity of saturated phosphatidylcholine-based liposomes,” Int J Pharmaceutics, vol. 483, pp. 142-150, 2015.

What is claimed is:

1. A method of preparing a liposomal composition comprising a water-insoluble or poorly water soluble compound, said method comprising (i) dissolving a water-insoluble or poorly water soluble compound in an alkaline aqueous solution at a final concentration of about 10-100 milligrams per milliliter; (ii) adding an amphiphilic lipid to said dissolved compound; (III) adding a protein to the dissolved compound; (iv) adding an edible oil to the dissolved compound; (v) high-shear mixing the dissolved compound with the addition of amphiphilic lipid, protein and edible oil; and (vi) neutralizing the mixture obtained in step (v).

2. The method according to claim 1, wherein the compound possesses one or more functional groups with a labile acidic hydrogen.

3. The method of claim 2, wherein the one of more functional groups are selected from the group consisting of carboxylate (carboxylic acid), enolate (enol), phenolate (phenol), α,β-unsaturated ketone, 1,3-diketone, thiolate (thiol), phosphate, phosphonate, ammonium, imide, or combinations thereof.

4. The method according to claim 1, wherein the compound is selected from the group consisting of alkaloids, carotenoids, anthocyanidins, catechins, flavanols, flavonoids, phytosterols, polyphenols, or combinations thereof.

5. The method according to claim 1, wherein the compound is selected from the group consisting of herbal extract or isolate, fruit or vegetable extract or isolate, vitamin or metabolic isolate, or combinations thereof.

6. The method of claim 5, wherein the herbal extract or isolate is ashwagandha, boswellia or boswellic acids, curcumin or curcuminoids, cannabidiol (CBD) or related cannabinoids, ginseng or ginsenosides, sage, gingko biloba, ginger, St. John's Wort or hypericin, epigallocatechin gallate (EGCG), turmeric oil, or combinations thereof.

7. The method of claim 5, wherein the fruit or vegetable extract or isolate is astaxanthin, beta carotene, fisetin, quercetin, resveratrol, sulforaphane, avocado or soybean unsaponifiables, or combinations thereof.

8. The method of claim 5, wherein the vitamin or metabolic isolate is biotin, Vitamin D, Vitamin K, coenzyme Q10 (CoQ10), glutathione, retinoic acid, ubiquinol, ubiquinone, or combinations thereof.

9. The method according to claim 1, wherein the aqueous solution is made alkaline by adding bases or basic compounds selected from the group consisting of sodium hydroxide, potassium hydroxide, lithium hydroxide, ammonium hydroxide, sodium carbonate, potassium carbonate, calcium carbonate, sodium bicarbonate, potassium bicarbonate, or combinations thereof.

10. The method according to claim 1, wherein the amphiphilic lipid is selected from the group consisting of phospholipids, soy lecithin, sunflower lecithin, rapeseed lecithin, egg yolk lecithin, canola lecithin, cottonseed lecithin, phosphatidylcholine and derivatives, phosphatidylinositol and derivatives, phosphatidylethanolamine and derivatives, phosphatidylserine and derivatives, phosphatidylglycerol and derivatives, phosphatidic acid and derivatives, sphingolipids, glycosphingolipids, sphingomyelin and derivatives, or combinations thereof.

11. The method of claim 10, wherein the phosphatidylcholine is distearoylphosphatidylcholine or dimyristoylphosphatidylcholine.

12. The method of claim 10, wherein the phosphatidylglycerol is dimyristoylphosphatidylglycerol.

13. The method according to claim 1, wherein the protein is selected from the group consisting of whey protein, bone broth, pea protein, sunflower protein, pumpkin seed protein, soybean protein, flaxseed protein, hemp protein, chia seed protein, egg white protein, egg yolk protein, brown rice protein, ovalbumin, casein, bovine serum albumin, collagen, gelatin, or combinations thereof.

14. The method according to claim 1, wherein the protein is added at a concentration of about 0.1% to about 5.0% protein.

15. The method according to claim 1, wherein the edible oil is selected from the group consisting of sunflower oil, canola oil, safflower oil, soybean oil, corn oil, olive oil, peanut oil, almond oil, flaxseed oil, grapeseed oil, hemp oil, chia seed oil, avocado oil, Ahiflower oil, fish oil, krill oil, medium chain triglyceride (MCT) oil, or combinations thereof.

16. The method according to claim 1, wherein the edible oil is added at a concentration of about 0.5% to about 20.0% edible oil.

17. The method according to claim 1, further comprising neutralizing the liposomal composition obtained in step (v) with an acid or an acidic compound selected from the group consisting of hydrochloric acid, sulfuric acid, nitric acid, acetic acid, carbonic acid, citric acid, ascorbic acid, or combinations thereof.

18. The method according to claim 17, wherein the liposomal composition obtained in step (v) is acidified to a pH of about 4.6 or less.

19. The method of claim 1 further comprising adding a polysaccharide gum to the liposomal composition selected from the group consisting of locust bean gum, guar gum, gellan gum, xanthan gum, pectin, gum Arabic, agar-agar, carrageenan, alginates, tapioca, methylcellulose, carboxymethylcellulose, cornstarch, potato starch, other starches, or combinations thereof.

20. A method of preparing a liposomal composition comprising a water-insoluble or poorly water soluble compound, said method comprising (i) dissolving a water-insoluble or poorly water soluble compound in an acidic aqueous solution at a final concentration of about 10-100 milligrams per milliliter; (ii) adding an amphiphilic lipid to said dissolved compound; (III) adding a protein to the dissolved compound; (iv) adding an edible oil to the dissolved compound; (v) high-shear mixing the dissolved compound with the addition of amphiphilic lipid, protein and edible oil; and (vi) neutralizing the mixture obtained in step (v).

21. The method according to claim 20, wherein the compound possesses one or more functional groups to which a labile acidic hydrogen can be added.

22. The method of claim 21, wherein the one of more functional groups are selected from the group consisting of amide, imide, amine, pyrrolidine, piperidine, pyridine, indole, imidazole, purine, or combinations thereof.

23. The method according to claim 20, wherein the compound is selected from the group consisting of alkaloids, carotenoids, anthocyanidins, catechins, flavanols, flavonoids, phytosterols, polyphenols, or combinations thereof.

24. The method according to claim 20, wherein the compound is selected from the group consisting of a herbal extract or isolate, a fruit or vegetable extract or isolate, a vitamin or metabolic isolate, or combinations thereof.

25. The method of claim 24, wherein the herbal extract or isolate is ashwagandha, berberine, Boswellia, theobromine, ginseng or ginsenosides, sage, gingko biloba, ginger, St. John's Wort, hypericin, turmeric oil, or combinations thereof.

26. The method of claim 24, wherein the fruit or vegetable extract or isolate is astaxanthin, beta carotene, fisetin, quercetin, resveratrol, sulforaphane, avocado or soybean unsaponifiables, or combinations thereof.

27. The method of claim 24, wherein the vitamin or metabolic isolate is biotin, niacinamide, thiamine, glutathione, or combinations thereof.

28. The method according to claim 20, wherein the aqueous solution is made acidic by adding acids or acidic compounds selected from the group consisting of hydrochloric acid, sulfuric acid, nitric acid, acetic acid, carbonic acid, citric acid, ascorbic acid, or combinations thereof.

29. The method according to claim 20, wherein the amphiphilic lipid is selected from the group consisting of phospholipids, soy lecithin, sunflower lecithin, rapeseed lecithin, egg yolk lecithin, canola lecithin, cottonseed lecithin, phosphatidylcholine and derivatives, phosphatidylinositol and derivatives, phosphatidylethanolamine and derivatives, phosphatidylserine and derivatives, phosphatidylglycerol and derivatives, phosphatidic acid and derivatives, sphingolipids, glycosphingolipids, sphingomyelin and derivatives, or combinations thereof.

30. The method of claim 29, wherein the phosphatidylcholine is distearoylphosphatidylcholine or dimyristoylphosphatidylcholine.

31. The method of claim 29, wherein the phosphatidylglycerol dimyristoylphosphatidylglycerol.

32. The method according to claim 20, wherein the protein is selected from the group consisting of whey protein, bone broth, pea protein, sunflower protein, pumpkin seed protein, soybean protein, flaxseed protein, hemp protein, chia seed protein, egg white protein, egg yolk protein, brown rice protein, ovalbumin, casein, bovine serum albumin, collagen, gelatin, or combinations thereof.

33. The method according to claim 20, wherein the protein is added at a concentration of about 0.1% to about 5.0% protein.

34. The method according to claim 20, wherein the edible oil is selected from the group consisting of sunflower oil, canola oil, safflower oil, soybean oil, corn oil, olive oil, peanut oil, almond oil, flaxseed oil, grapeseed oil, hemp oil, chia seed oil, avocado oil, Ahiflower oil, fish oil, krill oil, medium chain triglyceride (MCT) oil, or combinations thereof.

35. The method according to claim 20, wherein the edible oil is added at a concentration of about 0.5% to about 20.0% edible oil.

36. The method according to claim 20, further comprising neutralizing the liposomal composition obtained in step (v) with bases or basic compounds selected from the group consisting of sodium hydroxide, potassium hydroxide, lithium hydroxide, ammonium hydroxide, sodium carbonate, potassium carbonate, calcium carbonate, sodium bicarbonate, potassium bicarbonate, or combinations thereof.

37. The method according to claim 36, wherein the liposomal composition obtained in step (v) is neutralized to a pH of about 4.6 or less.

38. The method of claim 20 further comprising adding a polysaccharide gum to the liposomal composition, wherein the polysaccharide gum is selected from the group consisting of locust bean gum, guar gum, gellan gum, xanthan gum, pectin, gum Arabic, agar-agar, carrageenan, alginates, tapioca, methylcellulose, carboxymethylcellulose, cornstarch, potato starch, other starches, or combinations thereof.

39. The method according to claim 1, wherein the resulting liposomal composition is dried and ground to a fine powder.

40. The method according to claim 20, wherein the resulting liposomal composition is dried and ground to a fine powder.
